(19) United States
(12) Patent Application Publication
(10) Pub. No.: US 2009/0120444 A1
(43) Pub. Date: May 14, 2009

(54) Single-Lumen Endotracheal Tube for Partial Lung Ventilation

(76) Inventor: José Carlos Romeiro Sapienza, Rio de Janeiro (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/813,353

(22) PCT Filed: Jan. 5, 2005
(86) PCT No.: PCT/BR05/00001

§ 371 Date: Apr. 15, 2008

(51) Int. Cl. A61M 16/04

(52) U.S. Cl. 128/207.15

(57) ABSTRACT

An endotracheal tube is provided with a catheter (5) that extends outwards and beyond a point that coincides with the beginning of the chamfered region of the tube (1) at its distal or inner end (3). In the region of the distal end of the catheter (5), the latter is provided with an inflatable ballonet (6). With this tube inserted until its distal end (3) is located inside the trachea, the catheter (5) enters the bronchus to be obstructed. The ballonet at the distal end of the catheter may then be inflated, obstructing that bronchus. According to the invention, the chamfered region of the distal end (3) of the tube (1) comprises a smooth tube wall without any lateral opening, which does not deviate from the axis of the tube, with respect to the wall of the tube (1) immediately prior to the chamfered region.

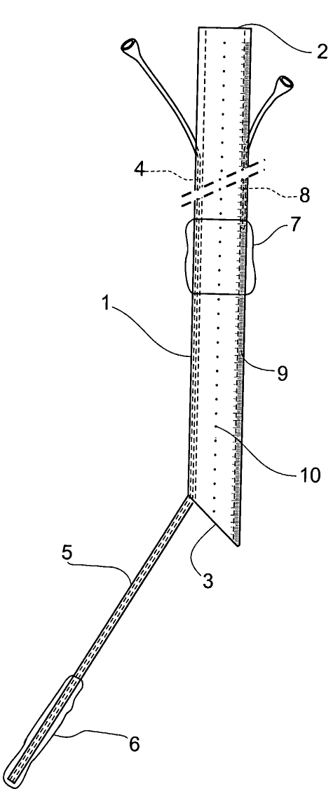

The present invention relates to a single-lumen endotracheal tube for use in anesthesia and other procedures that require ventilation or selective blocking of the lungs.

### PRIOR ART AND ITS DRAWBACKS

Traditional procedures use, on the one hand, CARLENS-type or ROBERTSHAW-type endotracheal tubes, which have the disadvantage of having a double-lumen which, due to their dimensions, prevents their pediatric use and, on the other hand, the previous selective blocking of the bronchus to be obstructed, using movable catheters with inflatable ROGARTY-type ballonets. In the latter case, the use of bronchoscopy apparatuses becomes compulsory.

The known drawbacks of these traditional procedures have been solved by using a single-lumen endotracheal tube, as disclosed in Brazilian Patent PI 9005721-0. The endotracheal tube described and illustrated in that patent has an outer end and a chamfered distal region that ends in a distal end of the tube. Integrated with the tube, there is a catheter that extends outwards and past the point that coincides with the beginning of the chamfered region of the tube as far as a distal catheter end that is provided with an inflatable catheter ballonet. With this tube inserted until its distal end is located within the trachea to be ventilated, the catheter extends into the main bronchus (to be obstructed). The ballonet at the distal end of the catheter is then inflated, obstructing that main bronchus.

Although the endotracheal tube of Brazilian patent PI 9005721-0has solved the basic drawbacks associated with the traditional procedures, another problem has arisen. The distal end of the tube, designed for remaining in the bronchus, was laterally diverted with respect to its axis, forming a spur, which in turn has a so-called “Murphy's eye” (a cutout at the end, forming a lateral opening that resembles an eye) to ensure ventilation and eliminate any risk of obstruction of the bronchus to be ventilated. This diversion of about 10 degrees at the end of the tube may result in interference with the side wall of the trachea, rendering it difficult to insert the tube and/or injuring the trachea itself. This is particularly relevant in pediatric use, when the tracheas are diminutive.

### THE PRESENT INVENTION

According to the present invention, a single-lumen endotracheal tube for selective ventilation of the type described in Brazilian patent PI 9005721-0 is characterized in that at least the chamfered region of the distal end of the tube comprises a smooth tube wall without any lateral opening, which does not deviate from the tube axis, with respect to the tube wall immediately before the chamfered region.

Surprisingly, the absence of the lateral spur with the “Murphy's eye” does not at all impair the safety of the device, since the absence of deviation from the axis of the tube, combined with the chamfered region, will result in easy reduction and positioning of the tube in the trachea, without the need for security side ventilation.

In addition, according to a secondary aspect of the invention, however, the catheter ballonet will occupy an extent of about 40% of the catheter length, which projects beyond a point that coincides with the beginning of the chambered region. The tube according to the above-mentioned earlier patent used an inflatable ballonet that occupied approximately 30% of the catheter length. With the increase to approximately 40%, not only is there greater efficiency in its action of effectively obstructing the respective main bronchus, mainly in the right lung, but also a firmer positioning of the tube, further reducing the displacement of the distal end of the tube in the bronchus being ventilated.

Another optional characteristic of the present invention, of important utility in practice, is the fact that the outer wall of the tube is provided with graduation marks between the distal end of the tube and its external end. Thus, an anesthetist can precisely control the degree of introduction of the tube into the bronchus.

### BRIEF DESCRIPTION OF THE DRAWING

The present invention will now be described in greater detail, by way of example, with reference to the single drawing attached hereto, which is a side view of a single-lumen endotracheal tube for lung ventilation according to the presently preferred embodiment of the present invention.

### DETAILED DESCRIPTION OF THE DRAWING

The FIGURE shows an endotracheal tube according to the preferred embodiment of the invention, comprising a polyvinyl or similar rubber tube 1, which has an outer or upper end 2 and a chamfered or beveled end 3. Within tube 1, in its side wall, there is an air-blowing channel 4 which continues, at the beginning of chambered end 3, in the form of a catheter 5 with an inflatable ballonet 6 at its distal end.

The catheter 5 is inclined by approximately 30 degrees with respect to the axis of the tube 1 and the ballonet 6 occupies approximately 40% of the catheter extent, finishing at the distal end of the latter.

An important characteristic of this invention is the fact that the chamfered end 3 of the tube 1 represents a continuation of the tube, without its wall being deviated or displaced to either side. The tube wall at the chamfered end 3 is smooth and without openings (there is no “Murphy's eye”). In reality, the chamfered end is a mere continuation of tube 1, without any other modification than the inclined or chamfered cut end that makes an angle of approximately 30 degrees with the axis of the tube 1. The axial extent of the chamfer is about 20% of the catheter 5 length.

Further, around the tube 1 and in a generally middle region of its extent (preferably in the distal third part), there is a ballonet 7 that is inflatable by means of a second blowing channel 8 formed inside the tube wall.

Along the tube 1 there is a graduation 9 (in centimeters) that begins at the tip of the chamfering of the end 3 and continues as far as the outer end 2 of the tube, thus enabling the surgeon to determine in accuracy the degree of introduction of the device. Another graduation is also foreseen and indicated by reference number 10, in reference units equal to the axial extent of the chamfering of the tube end 3, this latter varying according to the age of the patient (adult, bigger child, smaller child, baby, etc.).

In use, tube 1 is introduced as far as its forked distal end, as formed by the chamfered end 3 and the bronchus catheter 5, straddled over the tracheal carina. Then the tube is pulled/withdrawn for the equivalent of two of the graduations 10, that is to say, twice the axial dimension of the chamfering. The fact that there is no axial deviation along the length of tube 1, nor any spur or “Murphy's eye”, facilitates the introduction of the device without risk of injuring the tracheal wall. Once the correct position has been found, the surgeon inflates the ballonet 7 through channel 8 to hold the device in place, with the two bronchi duly ventilated. Then the ballonet 6 is inflated through the blowing channel 4, closing (obstructing) the respective bronchus, so that only the other bronchus remains ventilated through tube 2. With the two ballonets 6 and 7 inflated, the device remains stable in place, and the ventilation of the other bronchus is maintained.

1. A single-lumen endotracheal tube, comprising a tube having an outer end and a chamfered distal region that ends at a distal end of the tube, and a catheter integrated with the tube, the catheter extending from a point that coincides with the beginning of said chamfered region of the tube as far as a distal end of the catheter that is provided with an inflatable catheter ballonet, characterized in that:
at least the chamfered region of the distal end of the tube comprises a smooth tube wall without any lateral opening, which does not deviate from the axis of the tube, with respect to the tube wall immediately before the chamfered region; and
said catheter is angled away from said tube whereby the catheter and said chamfered region together define a forked distal end of said tube.

2. An endotracheal tube according to claim 1, characterized in that said catheter is angled away from said tube by approximately 30° with respect to the axis of said tube.

3. An endotracheal tube according to claim 1, characterized in that the inflatable ballonet of the catheter occupies an extent of approximately 40% of the catheter length that projects beyond said point that coincides with the beginning of the chamfered region of the tube.

4. An endotracheal tube according to claim 1, characterized in that the outer wall of the tube is provided with graduation markings between the distal end of the tube and its outer end.
